(19) United States
(12) Patent Application Publication
(10) Pub. No.: US 2005/0032690 A1
(43) Pub. Date: Feb. 10, 2005

(54) Factor VII polypeptides for preventing formation of inhibitors in subjects with haemophilia

(76) Inventors: Lisa Payne Rojkjaer, Gentofte (DK); Marie Johannessen, Birkerod (DK); Ole Juul Nordfang, Hillerod (DK); Jens Aas Jansen, Charlottenlund (DK)

Correspondence Address: Reza Green, Esq.;Novo Nordisk Pharmaceuticals, Inc., 100 College Road West, Princeton, NJ, 08540, US

(21) Appl. No.: 10/737,936

(22) Filed: Dec. 17, 2003

Related U.S. Application Data
(63) Continuation-in-part of application No. 10/026,032, filed on Oct. 25, 2001, now Pat. No. 6,833,352.
Application No. 10/026,032 is a continuation of application No. 09/148,440, filed on Sep. 4, 1998, now Pat. No. 6,310,183.
(63) Continuation-in-part of application No. 10/283,751, filed on Oct. 30, 2002.
(60) Provisional application No. 60/444,321, filed on Jan. 31, 2003.

(30) Foreign Application Priority Data
Sep. 10, 1997 (DK) 1038/97
May 2, 2001 (WO) PCT/DK01/00302
May 3, 2000 (DK) PA 2000 00734
Sep. 13, 2000 (DK) PA 2000 01360
Sep. 13, 2000 (DK) PA 2000 01361
Mar. 22, 2001 (DK) PA 2001 00477
Dec. 20, 2002 (DK) PA 2002 01989

(51) Int. Cl. A61K 38/38; A61K 38/10

(52) U.S. Cl. 514/12; 514/13; 514/14

(57) ABSTRACT

The invention provides a method for preventing formation of inhibitors to blood coagulation factor VIII or factor IX in a subject having haemophilia, the method comprising administering (via intravenous, subcutaneous, intradermal, or intramuscular routes) to a previously untreated subject an effective dosage of factor VIIa or a factor VII-related polypeptide.

### CROSS-REFERENCE TO RELATED APPLICATIONS

This application is a non-provisional application of U.S. provisional application Ser. No. 60/444,321, filed Jan. 31, 2003; a continuation-in-part of pending U.S. application Ser. No. 10/026,032, filed Oct. 25, 2001 (which is a continuation of U.S. application Ser. No. 09/148,440, filed Sep. 4, 1998, now U.S. Pat. No. 6,310,183); and a continuation in part of pending U.S. application Ser. No. 10/283,751, filed Oct. 30, 2002, the contents of all of which are incorporated herein by reference in their entirety.

### FIELD OF THE INVENTION

The invention relates to the field of haemophilia. The invention provides methods for prevention of inhibitors to coagulation factors VIII or IX in previously untreated subjects having haemophilia.

### BACKGROUND OF THE INVENTION

Blood coagulation factor VII (FVII) is a plasma coagulation factor. Activated factor VII (FVIIa) initiates the normal haemostatic process by forming a complex with tissue factor (TF), exposed as a result of the injury to the vessel wall, which subsequently activates factors 1× and X (FIX and FX) into their activated forms, factors IXa and Xa (FIXa and FXa). Factor Xa converts limited amounts of prothrombin to thrombin on the tissue factor-bearing cell. Thrombin activates platelets and factors V and VIII into factors Va and VIIIa (FVa and FVIIIa), both cofactors in the further process leading to the full thrombin burst. This process includes generation of factor Xa by factor IXa (in complex with factor VIIIa) and occurs on the surface of activated platelets. Thrombin finally converts fibrinogen to fibrin resulting in formation of a fibrin clot.

Factor VII exists in plasma mainly as a single-chain zymogen, which is cleaved by FXa into its two-chain, activated form, FVIIa. Recombinant activated factor VII (rFVIIa) has been developed as a pro-haemostatic agent. The administration of rFVIIa offers a rapid and highly effective pro-haemostatic response in haemophilic subjects with bleedings who cannot be treated with coagulation factor products due to antibody formation. Also bleeding subjects with factor VII deficiency or subjects having a normal coagulation system but experiencing excessive bleeding can be treated successfully with FVIIa. In these studies, no unfavourable side effects of rFVIIa (in particular the occurrence of thromboembolism) has been encountered.

Blood coagulation factor VIII is a glycoprotein (MW 330,000) that circulates in blood. It is secreted by the liver and the endothelium and secreted into plasma where it circulates as a complex with von Willebrand factor. Factor VIII functions as a cofactor in blood coagulation in that it accelerates the conversion of factor X to factor Xa in the presence of factor IXa, calcium and phospholipid. Even though it is synthesized as a single polypeptide chain, it circulates in plasma primarily as a two-chain molecule. Activation of FVIII into an active cofactor requires additional proteolysis by thrombin or some other protease. A decrease in the presence or activity of factor VIII in the blood stream leads to haemophilia A. The level of the decrease in factor VIII activity is directly proportional to the severity of the disease. The current treatment of haemophilia A consists of the replacement of the missing protein by plasma-derived or recombinant factor VIII (so-called FVIII substitution or replacement treatment or therapy).

Blood coagulation factor IX (factor IX) is a plasma coagulation factor participating in the activation of factor X (FX). A decrease in the presence or activity of Factor IX in the blood stream leads to haemophilia B. The level of the decrease in Factor IX activity is directly proportional to the severity of the disease. The current treatment of haemophilia B consists of the replacement of the missing protein by plasma-derived or recombinant factor IX (so-called FIX substitution or replacement treatment or therapy).

Coagulation factor deficiencies (e.g., deficiency in factors VIII or IX) reflect different types of gene defects. Where the genetic lesion is severe, such as, deletion or frame shift, mRNA is not produced and (severe) deficiency results. Less severe genetic lesions from, for instance, point mutations which are not critically located result in secretion of protein with reduced biological activity. The inheritance patterns are recessive and X-linked, meaning that usually only men having one X-chromosome are affected. The severity of the coagulation defects can be mild or severe. Severity depends on the concentration of normally functioning factor VIII or factor IX in plasma. The aim of factor replacement therapy is to raise the level of the patient's clotting factor activity (hereinafter called the “factor level”) to one that will bring around haemostasis and to maintain it until healing is substantially complete. If the initiation of effective treatment is delayed, wound healing may be impaired and more factor replacement than usual will be required. The amount of factor replacement depends upon the plasma concentration of the coagulation factor needed for haemostasis, the recovery in blood and the half-life of the transfused material.

The level of factor VIII or factor IX may also be more or less reduced in some subjects (e.g., women being carriers of the disease) who are heterozygous for the gene defect. Such subjects may have an increased bleeding tendency comparable to that of mildly-affected haemophilia patients and may be treated accordingly.

Some patients receiving factor VIII/factor IX replacement therapy (having haemophilia A or B) develop antibodies against the administered factor VIII/factor IX. As well, persons born with a normal factor VIII/factor IX level may for unknown reasons later in life develop auto-antibodies against factor VIII/factor IX (acquired haemophilia A or B). In both cases the antibodies may be present in low, medium or high titres. In case of patients having a low or medium inhibitor-titre, these may sometimes be treated with factor VIII or factor IX, respectively.

Haemophilia occurs in all degrees of severity. The patient with less than 1% factor VIII/factor IX is severely affected and bleeds into muscles and joints with minimal trauma and sometimes spontaneously. A small amount of factor VIII/factor IX gives considerable protection so that patients with 1-5% of normal level factor VIII/factor IX usually suffer only posttraumatic bleeding and less severe bleeding into muscles and joints, etc., and are classified as having moderate haemophilia. Patients with more than 5% of factor VIII/factor IX usually bleed only after trauma or surgery and are classified as having mild haemophilia. It must be realised that the clinical symptoms can vary. Some patients with very low factor VIII/factor IX levels rarely bleed whilst others even with over 5% factor VIII/factor IX may bleed repeatedly into a “target joint” damaged originally by a traumatic haemarthrosis. As a generalisation, however, bleeding symptoms are less obvious with higher factor levels so that abnormal bleeding does not usually occur at factor VIII/factor IX levels over 35-40% of normal level. The general correlation between factor levels and symptoms in haemophilia A or B is shown below.

Severity of haemophilia related to factor VIII/factor IX levels:

| | Factor | |
|---|---|---|
| | Level (% of | |
| Severity | normal level) | Type of presentation |
| Severe | <1 | Bleeds with minimal or no obvious injury . . . |
| | | Severe bleeding |
| Moderate | 1-5 | Few bleeds. Haemarthroses mainly trauma- |
| | | induced |
| Mild | >5 | Post-traumatic, post-surgical, post-dental |
| | | extraction bleeding. Few episodes. |

The current treatment of haemophilia A or B consists of the replacement of the missing coagulation factor by plasma-derived or recombinant factor VIII or factor IX, respectively. Factor VIII/factor IX products are used as I.V. infusion (or injection) to treat acute bleeds on demand, or to prevent bleeding in association with surgery. The bleeding types are categorised as follows:
1. Mild to moderate bleeds (include soft tissue, muscle, joint/target joint bleeds)
2. Life and limb threatening bleeds (retroperitoneal bleeds, CNS bleeds, retropharyngeal bleeds, muscular bleeds with compartment syndrome and massive. GI bleeds)
3. Bleeding prevention in relation to surgery (orthopaedic, elective procedures, emergency surgery), or trauma.

Experience has shown that if factor VIII/factor IX levels are maintained over 30-40% of normal level until healing is complete then normal haemostasis is usually maintained. However other considerations are also important. Movement of the affected parts after surgery may promote bleeding. Physiotherapy or manipulation may require rather high factor levels whilst immobilisation of mild lesions may allow control of bleeding with relatively low factor levels. Approximate target factor levels which can be aimed for in various situations are shown below:

Haemophilia A:

Treatment of Mild to Moderate Bleeds (Category 1):

The goal is to achieve a factor VIII plasma concentration of 30-50% of normal level as needed.

Treatment of Life-and Limb Threatening Bleeds (Category 2):

The goal is to achieve an initial factor VIII plasma concentration of 100% initially, followed by a plasma concentration of 50-100% for 10-14 days.

Bleeding Prevention in Relation to Surgery or Trauma (Category 3):

The goal is to achieve a factor VIII plasma concentration of at least 100% on the day of surgery followed by a plasma concentration of 50% until wound healing begins (day 2 to 7), then-30% until wound healing is complete (one to two weeks).

In clinical treatment of haemophilia, factor VIIIa is presently used to stop bleedings in patients having inhibitors to FVIII or FIX, as inhibitors make standard factor replacement therapy ineffective. However, clinicians do not normally use factor VIIa as first line treatment for haemophiliacs without inhibitors because it is expected that the short half-life of factor VIIIa (2.5 hours) compared to that of factor VIII (10-12 hours) and factor IX (18-24 hours) would require more frequent factor VIIIa injections to maintaining a certain level of haemostatic ability.

European Patent No. 225.160 (Novo Nordisk) concerns compositions of FVIIa and methods for the treatment of bleeding disorders not caused by clotting factor defects or clotting factor inhibitors.

European Patent No. 82.182 (Baxter Travenol Lab.) concerns a composition of factor VIIa for use in counteracting deficiencies of blood clotting factors or the effects of inhibitors to blood clotting factors in a subject.

Lusher et al., Haemophilia, 1998, 4, pp. 790-798 concerns the administration of recombinant factor VIIa in treatment of joint, muscle and mucotaneous haemorrhages in persons with haemophilia A and B, with and without inhibitors.

Today, many recombinant factor VIII products are used in treatment of haemophilia. However, some plasma-derived (pd) factor products are still used therapeutically (pdFVIII, pdFIX, activated prothrombin complex concentrates [aPCCs], porcine FVIII), some of which contain lesser amounts of other coagulation factors or other components from plasma (aPCCs). Although rare, there is a risk of human or porcine viral transmission with plasma-derived products, and thus the use of recombinant factors is preferable as there is no such risk.

Today, subjects having a reduced level of factor VIII or factor IX experiencing bleeding episodes are generally treated with substitution of the missing protein. In a considerable number of cases, this treatment, however, results in formation of inhibitors, or antibodies, to the substituted protein. This formation of inhibitors, in turn, renders the treatment with factor VIII or factor IX less effective or even useless which leaves such patients with only a few feasible treatments, including treatment with recombinant factor VIIa, in case of bleeding episodes.

There is a need to develop strategies to prevent formation of inhibitors to factors VIII or IX in subjects having haemophilia because inhibitors result in much higher treatment costs for the patient and increased morbidity. As well, the “gold standard” of inhibitor management currently is immune tolerance therapy (ITT), which is a very rigorous undertaking for the very young patient and his family, has a high incidence of complications, some of which result in prolonged hospitalization, a high withdrawal rate, and the costs of factor replacement only for ITT can exceed 1 million USD per year. Such prevention or delay formation of inhibitors will be considered a definite improvement in the treatment and quality of life of the patient.

### SUMMARY OF THE INVENTION

The present invention provides a method for preventing formation of inhibitors to blood coagulation factors VIII or IX in a subject having haemophilia, the method comprising administering to a previously untreated subject an effective dosage of factor VIIa or a factor V11-related polypeptide.

In different embodiments thereof, the administered dosage is at least 120 microg/kg bw factor VIIa, such as at least 150 microg/kg factor VIIIa or at least 180 microg/kg, or a corresponding dose of a factor VII-related polypeptide. In different embodiments, factor VIIIa may be administered intravenously. In other embodiments, factor VIIa may be administered subcutaneously, intradermally, or intramuscularly.

In different embodiments, the polypeptide is human factor VIIa; or a factor VII-related polypeptide. In one embodiment, the polypeptide is a factor VII sequence variant, wherein the ratio between the activity of said factor VII polypeptide and the activity of native human factor VIIIa (wild-type FVIIa) is at least about 1.25 when tested in an “in Vitro Hydrolysis Assay” (such as, e.g., as described herein below.).

In one embodiment. the factor VII-related polypeptide is selected from S52A-FVIIa, S60A-FVIIa, FVIIa variants exhibiting increased proteolytic stability as disclosed in U.S. Pat. No. 5,580,560; Factor VIIa that has been proteolytically cleaved between residues 290 and 291 or between residues 315 and 316; oxidized forms of Factor VIIIa; L305V-FVII, L305V/M306D/D3095-FVII, L305-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII; L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/N158D/K337A/M298Q-FVII, L305V/V158D/E296V/K337A-FVII, L305V/V158D/E296V/M298Q/K337A-FVII, L305V/V158T/E296V/M298Q/K337A-FVII; S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V1158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/N158T/K337A/M298Q-FVII, S314E/L305V/V158T/E296V/K337A-FVII, S314E/L305V/V158D/K337A/M298Q-FVII, S314E/L305V/V158D/E296V/K337A —FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305H/V296V-FVII, K316H/L305V/M298Q-FVII, K316H/L305V/V158T-FVII, K316H/L305V/K337A/V1158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/V296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A —FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/V296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/V296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A —FVII, K316Q/L305V/V158D/E296V/M298Q/K337A-FVII, and K316Q/L305V/V158T/E296V/M298Q/K337A-FVII.

In different embodiments, the subject has haemophilia A or the subject has haemophilia B.

In one embodiment, the administered dosage is in the range of from about 120 to 150 microg/kg bw of factor VIIIa or a corresponding dosage of a factor VII-related polypeptide.

In different embodiments, the previously untreated subject is below 36 months of age such as, e.g., below 24 months, 18 months, 12 months, 8 months, or 6 months of age.

In different embodiments, the subject is treated exclusively with factor VIIa until he or she has attained a critical age after which there is a diminished likelihood of developing antibodies to Factor VIII or Factor IX. In some embodiments, the critical age is 36 months of age. In other embodiments, the critical age is 24 months, 18 months, 12 months, or 6 months of age.

In one embodiment, the factor VIIIa or the factor VII-related polypeptide is administered in conjunction with a second hemostatic agent, such as, e.g., a component of the blood coagulation system. In these embodiments, factor VII or a factor VII-related polypeptide is administered in a first amount, and the second component is administered in a second amount, wherein the first and second amounts together are effective for treating a bleeding episode. Non-limiting examples of the second hemostatic agent include: factor XIII, factor V, PAI-1, factor XI, thrombomodulin, aprotinin, TAFI, a tPA-inhibitor, a TFPI-inhibitor, alpha2-antiplasmin, a protein C-inhibitor, a protein S-inhibitor, tranexamic acid, and epsilon-aminocaproic acid; in particular factor XIII, factor V, and PAI-1.

In one aspect, the invention makes it possible to administer Factor VIIIa subcutaneously, intramuscularly or intradermally, which provides an advantage for all patients in need of Factor VIIIa in using FVIIa for prophylactic treatment of haemophilia patients to avoid the risk of forming life threatening antibodies towards Factor VIII and Factor IX.

In another aspect, the invention provides the use of factor VIIIa or a factor VII-related polypeptide for the manufacture of a medicament for preventing formation of inhibitors to blood coagulation factors VIII or IX in a previously untreated subject with haemophilia.

### DETAILED DESCRIPTION OF THE INVENTION

The present invention provides methods and compositions for preventing the formation of inhibitors to blood coagulation factors VIII or IX by intravenous, subcutaneous, intradermal, or intramuscular administration of factor VIIIa to individuals who have not been previously treated with either factor VIII or factor IX in any form who are in need of prophylactic or therapeutic treatment of bleeding episodes.

Factor VII Polypeptides:

In practicing the present invention, any factor VII polypeptide may be used that is effective in preventing or treating bleeding. This includes factor VII polypeptides derived from blood or plasma, or produced by recombinant means.

The present invention encompasses factor VII polypeptides, such as, e.g., those having the amino acid sequence disclosed in U.S. Pat. No. 4,784,950 (wild-type human factor VII). In some embodiments, the factor VII polypeptide is human factor VIIa, as disclosed, e.g., in U.S. Pat. No. 4,784,950 (wild-type factor VII). In one series of embodiments, factor VII polypeptides include polypeptides that exhibit at least about 10%, preferably at least about 30%, more preferably at least about 50%, and most preferably at least about 70%, of the specific biological activity of human factor VIIIa. In one series of embodiments, factor VII polypeptides include polypeptides that exhibit at least about 90%, preferably at least about 100%, preferably at least about 120%, more preferably at least about 140%, and most preferably at least about 160%, of the specific biological activity of human factor VIIIa. In one series of embodiments, factor VII polypeptides include polypeptides that exhibit at least about 70%, preferably at least about 80%, more preferably at least about 90%, and most preferable at least about 95%, of identity with the sequence of wild-type factor VII as disclosed in U.S. Pat. No. 4,784,950.

As used herein, “factor VII polypeptide” encompasses, without limitation, factor VII, as well as factor VII-related polypeptides. The term “factor VII” is intended to encompass, without limitation, polypeptides having the amino acid sequence 1-406 of wild-type human factor VII (as disclosed in U.S. Pat. No. 4,784,950), as well as wild-type factor VII derived from other species, such as, e.g., bovine, porcine, canine, murine, and salmon factor VII, said factor VII derived from blood or plasma, or produced by recombinant means. It further encompasses natural allelic variations of factor VII that may exist and occur from one individual to another. Also, the degree and location of glycosylation or other post-translation modifications may vary depending on the chosen host cells and the nature of the host cellular environment. The term “Factor VIII” is also intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa.

“Factor VII-related polypeptides” include, without limitation, factor VII polypeptides that have either been chemically modified relative to human factor VII and/or contain one or more amino acid sequence alterations relative to human factor VII (i.e., factor VII variants), and/or contain truncated amino acid sequences relative to human factor VII (i.e., factor VII fragments). Such factor VII-related polypeptides may exhibit different properties relative to human factor VII, including stability, phospholipid binding, altered specific activity, and the like.

The term “factor VII-related polypeptides” is intended to encompass such polypeptides in their uncleaved (zymogen) form, as well as those that have been proteolytically processed to yield their respective bioactive forms, which may be designated “factor VIIIa-related polypeptides” or “activated factor VII-related polypeptides”

It further encompasses polypeptides with a slightly modified amino acid sequence, for instance, polypeptides having a modified N-terminal end including N-terminal amino acid deletions or additions, and/or polypeptides that have been chemically modified relative to human factor VIIIa.

Factor VII-related polypeptides, including variants, encompass those that exhibit at least about 10%, at least about 20%, at least about 25%, at least about 30%, at least about 40%, at least about 50%, at least about 60%, at least about 70%, at least about 75%, at least about 80%, at least about 90%, at least about 100%, at least about 110%, at least about 120%, or at least about 130%, of the specific activity of wild-type factor VIIa that has been produced in the same cell type, when tested in one or more of a clotting assay, proteolysis assay, or TF binding assay as described above.

In some embodiments the therapeutic polypeptides are factor VII-related polypeptides, in particular variants, wherein the ratio between the activity of said factor VII polypeptide and the activity of native human factor VIIIa (wild-type FVIIa) is at least about 1.25 when tested in an ‘In Vitro Hydrolysis Assay’ (see “Assays”, below); in other embodiments, the ratio is at least about 2.0; in further embodiments, the ratio is at least about 4.0. In some embodiments of the invention, the factor VII polypeptides are factor VII-related polypeptides, in particular variants, wherein the ratio between the activity of said factor VII polypeptide and the activity of native human factor VIIIa (wild-type FVIIa) is at least about 1.25 when tested in an “In Vitro Proteolysis Assay” (see “Assays”, below); in other embodiments, the ratio is at least about 2.0; in further embodiments, the ratio is at least about 4.0; in further embodiments, the ratio is at least about 8.0.

Non-limiting examples of factor VII variants having substantially the same or improved biological activity as wild-type factor VII include S52A-FVII, S60A-FVII (lino et al., Arch. Biochem. Biophys. 352: 182-192, 1998); L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII; FVIIa variants exhibiting increased proteolytic stability as disclosed in U.S. Pat. No. 5,580,560; factor VIIa that has been proteolytically cleaved between residues 290 and 291 or between residues 315 and 316 (Mollerup et al., Biotechnol. Bioeng. 48:501-505, 1995); and oxidized forms of factor VIIa (Kornfelt et al., Arch. Biochem. Biophys. 363:43-54, 1999). Non-limiting examples of factor VII variants having substantially reduced or modified biological activity relative to wild-type factor VII include R152E-FVIIa (Wildgoose et al., Biochem 29:3413-3420, 1990), S344A-FVIIa (Kazama et al., J. Biol. Chem. 270:66-72, 1995), FFR-FVIIa (Hoist et al., Eur. J. Vasc. Endovasc. Surg. 15:515-520, 1998), and factor VIIIa lacking the Gla domain, (Nicolaisen et al., FEBS Letts. 317:245-249, 1993). Non-limiting examples of chemically modified factor VII polypeptides and sequence variants are described, e.g., in U.S. Pat. No. 5,997,864.

The biological activity of factor VIIIa in blood clotting derives from its ability to (i) bind to tissue factor (TF) and (ii) catalyze the proteolytic cleavage of Factor 1× or Factor X to produce activated Factor 1× or X (Factor IXa or Xa, respectively).

For purposes of the invention, biological activity of factor VIIIa polypeptides (“factor VIIa biological activity”) may be quantified by measuring the ability of a preparation to promote blood clotting using factor VII-deficient plasma and thromboplastin, as described, e.g., in U.S. Pat. No. 5,997,864. In this assay, biological activity is expressed as the reduction in clotting time relative to a control sample and is converted to “factor VII units” by comparison with a pooled human serum standard containing 1 unit/ml factor VIIIa activity. Alternatively, factor VIIIa biological activity may be quantified by
(i) Measuring the ability of factor VIIa or a factor VIIIa-related polypeptide to produce activated Factor X (Factor Xa) in a system comprising TF embedded in a lipid membrane and Factor X. (Persson et al., J. Biol. Chem. 272:19919-19924, 1997);
(ii) Measuring Factor X hydrolysis in an aqueous system (“In Vitro Proteolysis Assay”, see below);
(iii) Measuring the physical binding of factor VIIIa or a factor VIIIa-related polypeptide to TF using an instrument based on surface plasmon resonance (Persson, FEBS Letts. 413:359-363, 1997); and
(iv) Measuring hydrolysis of a synthetic substrate by factor VIIIa and/or a factor VIIIa-related polypeptide (“In Vitro Hydrolysis Assay”, see below); and
(v) Measuring generation of thrombin in a TF-independent in vitro system.

The term “factor VII biological activity” or “factor VII activity” is intended to include the ability to generate thrombin; the term also includes the ability to generate thrombin on the surface of activated platelets in the absence of tissue factor.

A factor VIIIa preparation that may be used according to the invention is, without limitation, NovoSeven® (Novo Nordisk A/S, Bagsvaerd, Denmark).

Inhibitors:

As used herein, “inhibitor” refers to antibodies that form within a patient in response to the administration of exogenous factor VIII (FVIII) or factor IX (FIX). Detectable levels of alloantibodies to FVIII develop in approximately 31% of patients with severe haemophilia A after a mean of 9-12 exposures to FVIII. In haemophilia B patients, alloantibodies to FIX develop in 2-5% of patients after a mean of 10-12 exposures to FIX. The presence of inhibitors renders ineffective FVIII or FIX replacement therapy, respectively, resulting in increased treatment costs and morbidity for the patient.

A “naive” subject as used herein refers to a subject who has not previously received either or both of exogenous FVIII or FIX-containing compounds (plasma-derived or recombinant factor VIII or IX, cryoprecipitate, fresh frozen plasma (FFP), solvent-detergent treated plasma, porcine FVIII, whole blood, aPCCs/PCCs), for any reason.

The term “corresponding dose” or “corresponding amount” is meant to include, without limitation, an amount of a factor VII-related polypeptide that has an equivalent level of factor VIIIa biological activity as, e.g., at least 120 microg/kg factor VIIIa when tested in one or more of a clotting assay, proteolysis assay, or TF binding assay as described in the present specification (see “assay”-part, below). The term may, where appropriate, be used interchangeably with the term “equal dose” or “equal amount”.

In the present context the three-letter or one-letter indications of the amino acids have been used in their conventional meaning as indicated in table 1. Unless indicated explicitly, the amino acids mentioned herein are L-amino acids. It is to be understood, that the first letter in, for example, K337 represent the amino acid naturally present at the indicated position wild-type factor VII, and that, for example, K337A-FVIIa designates the FVII-variant wherein the amino acid represented by the one-letter code K naturally present in the indicated position is replaced by the amino acid represented by the one-letter code A.

TABLE 1
Abbreviations for amino acids:

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |

The terms “factor VII”, “Factor VII” or “FVII” may be used interchangeably. The terms “factor VIIIa”, “Factor VIIa” or “FVIIa” may be used interchangeably. The terms “factor VIII” or “Factor VIII” or “FVIII” may be used interchangeably. The terms “factor VIIIa” or “Factor VIIIa” or “FVIIIa” may be used interchangeably. The terms “factor IX” or “Factor IX” or “FIX” may be used interchangeably. The terms “factor IXa” or “Factor IXa” or “FIXa” may be used interchangeably.

Clot lysis time, clot strength, fibrin clot formation, and clotting time are clinical parameters used for assaying the status of patient's haemostatic system. Blood samples are drawn from the patient at suitable intervals and one or more of the parameters are assayed by means of, e.g., thromboelastograpy as described by, e.g., Meh et al., Blood Coagulation & Fibrinolysis 2001;12:627-637; Vig et al., Hematology, Vol. 6 (3) pp. 205-213 (2001); Vig et al., Blood coagulation & fibrinolysis, Vol. 12 (7) pp. 555-561 (2001) October; Glidden et al., Clinical and applied thrombosis/hemostasis, Vol. 6 (4) pp. 226-233 (2000) October; McKenzie et al., Cardiology, Vol. 92 (4) pp. 240-247 (1999) April; or Davis et al., Journal of the American Society of Nephrology, Vol. 6 (4) pp. 1250-1255 (1995).

As used herein the term “bleeding disorder” reflects any defect related to a reduced level of factor VIII or factor IX.

Bleeding refers to extravasation of blood from any component of the circulatory system. The term “bleeding episodes” is meant to include unwanted, uncontrolled and often excessive bleeding in subjects having bleeding disorders, e.g., in connection with surgery, trauma, or other forms of tissue damage, as well as bleedings in joints.

Bleedings may occur, e.g., in tissue, in joints, in organs such as the brain, inner ear region and eyes; these are areas with limited possibilities for surgical haemostasis and thus problems with achieving satisfactory haemostasis. Bleedings may also, for example, occur in the process of taking biopsies from various organs (liver, lung, tumour tissue, gastrointestinal tract) as well as in laparoscopic surgery and radical retropubic prostatectomy. Common for all these situations is the difficulty to provide haemostasis by surgical techniques (sutures, clips, etc.) which also is the case when bleeding is diffuse (e.g., haemorrhagic gastritis and profuse uterine bleeding). Bleeding episodes are also meant to include, without limitation, uncontrolled and excessive bleeding in connection with surgery or trauma in subjects having acute haemarthroses (bleedings in joints), chronic haemophilic arthropathy, haematomas, (e.g., muscular, retroperitoneal, sublingual and retropharyngeal, and secondary to injury or injection [vaccination]), bleedings in other soft tissues, compartment syndromes (existing or threatened), epistaxis (nose bleeds), haematuria (bleeding from the urogenital tract), cerebral haemorrhage, surgery (e.g., hepatectomy), dental extraction, circumcision, and gastrointestinal bleedings (e.g., UGI bleeds). The terms “bleeding episodes” and “bleedings” may, where appropriate, be used interchangeably.

In this context, the term “treatment” is meant to include both prevention of an expected bleeding, such as, for example, in major/minor surgery, and regulation of an already occurring bleeding, such as, for example, bleeding in a joint, or in trauma, with the purpose of inhibiting or minimising the bleeding. The above-referenced “expected bleeding” may be a bleeding expected to occur in a particular tissue or organ, or it may be an unspecified bleeding. Prophylactic administration of a preparation of factor VIII or a factor VII-related polypeptide is thus included in the term “treatment”.

The term “subject” as used herein is intended to mean any animal, in particular mammals, such as humans, and may, where appropriate, be used interchangeably with the term “patient”. By “level of factor VIII” or “factor VIII level” is meant the level of the patient's clotting factor VIII activity compared to the level in healthy subjects. The level is designated as a percentage of the normal level. The terms may, where appropriate, be used interchangeably.

“Haemophilia” refers to those subjects having bleeding symptoms due to a reduced plasma level/activity of factor VIII or factor IX, respectively.

By “reduced level of factor VIII” or “reduced factor VIII level” is meant a decrease in the presence or activity of Factor VIII in the blood stream compared to the mean factor VIII level in a population of subjects having no coagulation factor VIII deficiency or inhibitors to coagulation factor VIII. Based on its purification from human plasma, the concentration of factor VIII in the normal adult is about 100 to 200 ng/ml of plasma (mean value) which is equivalent to about 0.1 μM; this equivalents to 0.60-1.60 U/ml.

In normal healthy individuals, factor VIII activity and antigen levels vary between 60 and 160% of normal pooled plasma. Clinically, the level of circulating factor VIII can be measured by either a coagulant or an immunologic assay. Factor VIII procoagulant activity is determined by the ability of the patient's plasma to correct the clotting time of factor VIII-deficient plasma (e.g., an APTT assay, see below; see also “assay part” of the present description).

One unit of factor VIII has been defined as the amount of factor VIII present in one millilitre of normal (pooled) human plasma (corresponding to a factor VIII level of 100%).

One unit of factor VII is defined as the amount of factor VII present in 1 ml of normal plasma, corresponding to about 0.5 μg protein. After activation 50 units correspond to about 1 μg protein.

By “deficiency” is meant a decrease in the presence or activity of, e.g., factor VIII in plasma compared to that of normal healthy individuals. The term may, where appropriate, be used interchangeably with “reduced factor VIII level”.

By “APTT” or “aPTT” is meant the activated partial thromboplastin time (described by, e.g., Proctor RR, Rapaport SI: The partial thromboplastin time with kaolin; a simple screening test for first-stage plasma clotting factor deficiencies. Am J Clin Pathol 36:212, 1961).

By “level of factor IX” or “factor IX level” is meant the level of the patient's clotting factor IX activity compared to the level in healthy subjects. The level is designated as a percentage of the normal level. The terms may, where appropriate, be used interchangeably.

By “reduced level of factor IX” or “reduced factor IX level” is meant a decrease in the presence or activity of Factor IX in the blood stream compared to the mean factor IX level in a population of subjects having no coagulation factor IX deficiency or inhibitors to coagulation factor IX. Based on its purification from human plasma, the concentration of factor IX in the normal adult is about 300-400 microg/ml of plasma.

In normal healthy individuals, factor IX activity and antigen levels vary between 50 and 160% of normal pooled plasma. Clinically, the level of circulating factor IX can be measured by either a coagulant or an immunologic assay. Factor IX procoagulant activity is determined by the ability of the patient's plasma to correct the clotting time of factor IX-deficient plasma (e.g., in an APTT assay, see below; see also “assay part” of the present description).

One unit of factor IX has been defined as the amount of factor IX present in one millilitre of normal (pooled) human plasma (corresponding to a factor IX level of 100%).

One unit of factor VII is defined as the amount of factor VII present in 1 ml of normal (pooled) plasma, corresponding to about 0.5 μg protein. After activation 50 units correspond to about 1 μg protein.

By “deficiency” is meant a decrease in the presence or activity of, e.g., factor IX in plasma compared to that of normal healthy individuals. The term may, where appropriate, be used interchangeably with “reduced factor IX level”.

By “APTT” or “aPTT” is meant the activated partial thromboplastin time (described by, e.g., Proctor RR, Rapaport SI: The partial thromboplastin time with kaolin; a simple screening test for first-stage plasma clotting factor deficiencies. Am J Clin Pathol 36:212, 1961).

Abbreviations:

| | |
|---|---|
| TF | tissue factor |
| FVII | factor VII in its single-chain, unactivated form |
| FVIIa | factor VII in its activated form |
| rFVIIa | recombinant factor VII in its activated form |
| factor VIII | factor VIII in its zymogenic, unactivated form |
| factor VIIIa | factor VIII in its activated form |
| rfactor VIII | recombinant factor VIII |
| rfactor VIIIa | recombinant factor VIIIa |
| factor IX | factor IX in its zymogenic, unactivated form |
| factor IXa | factor IX in its activated form |
| rfactor IX | recombinant factor IX |
| rfactor IXa | recombinant factor IXa |

Dosage Range:

In practicing the present invention, it will be understood that any dosage of factor VIIIa may be used that is effective for treating bleeding episodes. Typically, the dosage range of the effective amount comprises from about 90 microg/kg bw factor VIIIa or a corresponding amount of a Factor VII-related polypeptide; in different embodiments, the dosage-effective amount comprises between about 120 and about 500 microg/kg; between 200 and about 500 microg/kg; between about 250 and about 500 microg/kg; between about 300 and about 500 microg/kg; between about 350 and 500 microg/kg; between about 400 and about 500 microg/kg; between about 450 and about 500 microg/kg; and greater than 500 microg/kg, respectively, of Factor VIIa or a corresponding amount of a Factor VII-related polypeptide. In one embodiment, the dosage-effective amount is between 120 and about 500 microg/kg; 120 and about 450 microg/kg; 120 and about 400 microg/kg; 120 and about 350 microg/kg; 120 and about 300 microg/kg; 120 and about 250 microg/kg; 120 and about 200 microg/kg; and 120 and about 150 microg/kg, respectively.

While FVIIa injected intravenously may be administered more frequently (such as, e.g., every second hour), FVIIa injected subcutaneously, intradermally or intramuscularly may be administered with an interval of 12-48 hours, preferably 24 hours. FVIIa may be administered by subcutaneous injections in an amount of about 100-100,000 units per kg body weight, and preferably in an amount of about 250-25,000 units per kg body weight corresponding to about 5-500 μg/kg.

In one aspect of the invention, patients are treated exclusively with factor VIIa until they have attained a critical age after which there is a diminished likelihood of developing antibodies to Factor VIII or Factor IX. In some embodiments, the critical age is 36 months of age. In other embodiments, the critical age is 24 months, 18 months, 12 months, or 6 months of age. The critical age for a particular type of patient may be determined, e.g., by applying conventional statistical analysis to retrospective studies involving administration of factor VIII or factor 1× to young children. It will be understood that any predictive method may be used, including, e.g., genotype analysis or any measurement that correlates with immune responsiveness to exogenously administered factor VIII or factor IX.

Combinatorial Treatment:

Factor VIIIa or a factor VIIIa-related polypeptide may also be administered in conjunction with a second hemostatic agent, such as, e.g., a component of the blood coagulation system. Non-limiting examples of such hemostatic agents include: factor XIII, factor V, PAI-1, factor XI, thrombomodulin, aprotinin, TAFI, a tPA-inhibitor, a TFPI-inhibitor, alpha2-antiplasmin, a protein C-inhibitor, a protein S-inhibitor, tranexamic acid, or epsilon-aminocaproic acid. For the purpose of the present invention, also variants such as, e.g., sequence variants, and derivatives such as, without limitation, truncated forms, or chemically derivatized forms of the respective polypeptides may be used if they retain the biological activity characteristic of the polypeptide from which they are derived. For example, variants or derivatives of factor XIII which have the kind of biological activity characteristic of factor XII may be used in the present invention. Assays for determining biological activity of factor XIII-, factor V-, PAI-1-, factor XI-, thrombomodulin-, aprotinin-, TAFI-, and alpha2-antiplasmin-related polypeptides as well biological activity associated with a tPA-inhibitor, a TFPI-inhibitor, a protein C-inhibitor, and a protein S-inhibitor are well-known in the art.

Preparation of Compounds:

Human purified factor VIIIa suitable for use in the present invention is preferably made by DNA recombinant technology, e.g. as described by Hagen et al., Proc.Natl.Acad.Sci. USA 83: 2412-2416, 1986, or as described in European Patent No. 200.421 (ZymoGenetics, Inc.).

Factor VII may also be produced by the methods described by Broze and Majerus, J. Biol. Chem. 255 (4): 1242-1247, 1980 and Hedner and Kisiel, J.Clin.Invest. 71: 1836-1841, 1983. These methods yield factor VII without detectable amounts of other blood coagulation factors. An even further purified factor VII preparation may be obtained by including an additional gel filtration as the final purification step factor VII is then converted into activated factor VIIIa by known means, e.g. by several different plasma proteins, such as factor XIIa, IX a or Xa. Alternatively, as described by Bjoern et al. (Research Disclosure, 269 September 1986, pp. 564-565), factor VI may be activated by passing it through an ion-exchange chromatography column, such as Mono Q® (Pharmacia fine Chemicals) or the like.

Factor VII-related polypeptides may produced by modification of wild-type factor VII or by recombinant technology. Factor VII-related polypeptides with altered amino acid sequence when compared to wild-type factor VII may be produced by modifying the nucleic acid sequence encoding wild-type factor VII either by altering the amino acid codons or by removal of some of the amino acid codons in the nucleic acid encoding the natural factor VII by known means, e.g. by site-specific mutagenesis.

It will be apparent to those skilled in the art that substitutions can be made outside the regions critical to the function of the factor VIIIa or factor VIII-molecule and still result in an active polypeptide. Amino acid residues essential to the activity of the factor VII or factor VII-related polypeptide or factor VIII or factor VIII-related polypeptide, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for coagulant, respectively cross-linking activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The introduction of a mutation into the nucleic acid sequence to exchange one nucleotide for another nucleotide may be accomplished by site-directed mutagenesis using any of the methods known in the art. Particularly useful is the procedure that utilizes a super coiled, double stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with Dpnl, which is specific for methylated and hemi-methylated DNA to digest the parental DNA template and to select for mutation-containing synthesized DNA. Other procedures known in the art for creating, identifying and isolating variants may also be used, such as, for example, gene shuffling or phage display techniques.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, factor VII or factor VII-related polypeptides may be further purified. Purification may be achieved using any method known in the art, including, without limitation, affinity chromatography, such as, e.g., on an anti-factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J. C. Janson and Lars Ryden, editors, VCH Publishers, New York, 1989. Following purification, the preparation preferably contains less than about 10% by weight, more preferably less than about 5% and most preferably less than about 1%, of non-factor VIII or factor VII-related polypeptides derived from the host cell.

Factor VII or factor VII-related polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, factor VII or factor VII-related polypeptides may be activated by passing it through an ion-exchange chromatography column, such as Mono Q® (Pharmacia) or the like. The resulting activated factor VII or factor VII-related polypeptide may then be formulated and administered as described below.

Factor VIII for use within the present invention may be isolated from plasma according to known methods, such as those disclosed, e.g., by Fulcher et al.; Proc. Acad. Nat. Sci. USA 1982; 79:1648-1652, and Rotblat et al.; Biochemistry 1985; 24:4294-4300. It is preferred, however, to use recombinant factor VIII so as to avoid to the use of blood- or tissue-derived products that carry a risk of disease transmission. Human purified Factor VIII suitable for use in the present invention is preferably made by DNA recombinant technology, e.g. as described by U.S. Pat. Nos. 4,757,006 and 4,965,199.

Factor VIII-related polypeptides may produced by modification of wild-type factor VIII or by recombinant technology. Factor VIII-related polypeptides with altered amino acid sequence when compared to wild-type factor VIII may be produced by modifying the nucleic acid sequence encoding wild-type factor VIII either by altering the amino acid codons or by removal of some of the amino acid codons in the nucleic acid encoding the natural factor VIII by known means, e.g. by site-specific mutagenesis, as described in more detail above. Separation of polypeptides from their cell of origin may be achieved by any method known in the art, including, without limitation, removal of cell culture medium containing the desired product from an adherent cell culture; centrifugation or filtration to remove non-adherent cells; and the like. Optionally, factor VIII or factor VIII-related polypeptides may be further purified. Purification may be achieved using any method known in the art, including, without limitation, affinity chromatography, such as, e.g., on an anti-factor VIII antibody column; hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like, as described in more detail above. Following purification, the preparation preferably contains less than about 10% by weight, more preferably less than about 5% and most preferably less than about 1%, of non-factor VIII or factor VIII-related polypeptides derived from the host cell. The resulting activated factor VIII or factor VIII-related polypeptide may then be formulated and administered as described below.

As will be appreciated by those skilled in the art, it is preferred to use factor VIII polypeptides and factor VII polypeptides syngeneic with the subject in order to reduce the risk of inducing an immune response. Preparation and characterization of non-human factor VIII has been disclosed by, for example, Fass et al.; Blood 1982; 59: 594-600. The present invention also encompasses the use of such factor VIII polypeptides and factor VII polypeptides within veterinary procedures.

Pharmaceutical Compositions and Methods of Use

Pharmaceutical compositions or formulations for use in the present invention comprise a Factor VIIIa preparation in combination with, preferably dissolved in, a pharmaceutically acceptable carrier, preferably an aqueous carrier or diluent. A variety of aqueous carriers may be used, such as water, buffered water, 0.4% saline, 0.3% glycine and the like. The preparations of the invention can also be formulated into liposome preparations for delivery or targeting to the sites of injury. Liposome preparations are generally described in, e.g., U.S. Pat. Nos. 4,837,028, 4,501,728, and 4,975,282. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

Loading and maintenance doses are, for example, administration of a loading dose of about 120 microg/kg bw of factor VIIIa and repeated doses every 2 to 3 hours if needed.

Assays:

Test for Factor VIIIa Activity:

A suitable assay for testing for factor VIIIa activity and thereby selecting suitable factor VIIIa variants can be performed as a simple preliminary in vitro test:

In Vitro Hydrolysis Assay

Native (wild-type) factor VIIIa and factor VIIIa variant (both hereafter referred to as “factor VIIIa”) may be assayed for specific activities. They may also be assayed in parallel to directly compare their specific activities. The assay is carried out in a microtiter plate (MaxiSorp, Nunc, Denmark). The chromogenic substrate D-Ile-Pro-Arg-p-nitroanilide (S-2288, Chromogenix, Sweden), final concentration 1 mM, is added to factor VIIa (final concentration 100 nM) in 50 mM Hepes, pH 7.4, containing 0.1 M NaCl, 5 mM CaCl₂ and 1 mg/ml bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme, is used to calculate the ratio between the activities of variant and wild-type factor VIIIa:
Ratio=(A₄₀₅ nm factor VIIIa variant)/(A₄₀₅₅ nm factor VIIIa wild-type).

Based thereon, factor VIIIa variants with an activity comparable to or higher than native factor VIIIa may be identified, such as, for example, variants where the ratio between the activity of the variant and the activity of native factor VII (wild-type FVII) is around, versus above 1.0.

The activity of factor VIIa or factor VIIa variants may also be measured using a physiological substrate such as factor X, suitably at a concentration of 100-1000 nM, where the factor Xa generated is measured after the addition of a suitable chromogenic substrate (eg. S-2765). In addition, the activity assay may be run at physiological temperature.

In Vitro Proteolysis Assay

Native (wild-type) Factor VIIIa and Factor VIIIa variant (both hereafter referred to as “Factor VIIIa”) are assayed in parallel to directly compare their specific activities. The assay is carried out in a microtiter plate (MaxiSorp, Nunc, Denmark). Factor VIIa (10 nM) and Factor X (0.8 microM) in 100 microL 50 mM Hepes, pH 7.4, containing 0.1 M NaCl, 5 mM CaCl2 and 1 mg/ml bovine serum albumin, are incubated for 15 min. Factor X cleavage is then stopped by the addition of 50 microL 50 mM Hepes, pH 7.4, containing 0.1 M NaCl, 20 mM EDTA and 1 mg/ml bovine serum albumin. The amount of Factor Xa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used to calculate the ratio between the proteolytic activities of variant and wild-type Factor VIIIa:

Ratio=(A405 nm Factor VIIIa variant)/(A405 nm Factor VIIIa wild-type).

Based thereon, factor VIIIa variants with an activity comparable to or higher than native factor VIIIa may be identified, such as, for example, variants where the ratio between the activity of the variant and the activity of native factor VII (wild-type FVII) is around, versus above 1.0.

Thrombin Generation Assay:

The ability of factor VII or factor VII-related polypeptides (e.g., variants) or factor 1× or factor IX-related polypeptides (e.g., variants) to generate thrombin can be measured in an assay comprising all relevant coagulation factors and inhibitors at physiological concentrations and activated platelets (as described on p. 543 in Monroe et al. (1997) Brit. J. Haematol. 99, 542-547 which is hereby incorporated as reference).

Test for Factor VIII Activity:

Suitable assays for testing for factor VIII activity, and thereby providing means for selecting suitable factor VIII variants for use in the present invention, can be performed as simple in vitro tests as described, for example, in Kirkwood T B L, Rizza C R, Snape T J, Rhymes Ill., Austen D E G. Identification of sources of interlaboratory variation in factor VIII assay. B J Haematol 1981; 37; 559-68.; or Kessels et al., British Journal of Haematology, Vol. 76 (Suppl. 1) pp. 16 (1990)). Factor VIII activity may also be measured by a two-step chromogenic assay based on the amidolytic activity of generated FXa (Wagenvoord et al, 1989, Haemostasis, 19(4):196-204) (“the chromogenic assay”).

Factor VIII biological activity may also be quantified by measuring the ability of a preparation to correct the clotting time of factor VIII-deficient plasma, e.g., as described in Nilsson et al., 1959.(Nilsson I M, Blombaeck M, Thilen A, von Francken I., Carriers of haemophilia A-A laboratory study, Acta Med Scan 1959; 165:357). In this assay, biological activity is expressed as units/ml plasma (1 unit corresponds to the amount of FVIII present in normal pooled plasma.

Test for Factor IX Activity:

Suitable assays for testing for factor IX activity, and thereby providing means for selecting suitable factor IX variants for use in the present invention, can be performed as simple in vitro tests as described, for example, in Wagenvoord et al., Haemostasis 1990;20(5):276-88 (“the chromogenic assay”)

Factor IX biological activity may also be quantified by measuring the ability of a preparation to correct the clotting time of factor IX-deficient plasma, e.g., as described in Nilsson et al., 1959.(Nilsson I M, Blombaeck M, Thilen A, von Francken I., Carriers of haemophilia A-A laboratory study, Acta Med Scan 1959; 165:357). In this assay, biological activity is expressed as units/ml plasma (1 unit corresponds to the amount of FIX present in normal pooled plasma.

1. A method for preventing formation of inhibitors to blood coagulation factor VIII, the method comprising administering to a naive subject in need of coagulation therapy a coagulation-effective amount of a polypeptide selected from the group consisting of factor VIIa and a factor VII-related polypeptide.

2. A method according to claim 1, wherein said administering is via an intravenous, subcutaneous, intradermal, or intramuscular route.

3. A method according to claim 1, wherein the amount is at least about 120 microg/kg.

4. A method according to claim 3, wherein the amount is at least about 150 microg/kg factor VIIa or a corresponding amount of a factor VIIa-related polypeptide.

5. A method according to claim 1, wherein the polypeptide is human factor VIIIa.

6. A method according to claim 1, wherein the factor VIIIa-related polypeptide is a factor VIIIa sequence variant.

7. A method according to claim 6, wherein the factor VIIIa-related polypeptide is selected from the group consisting of: S52A-FVIIa, S60A-FVIIa, FVIIa variants exhibiting increased proteolytic stability as disclosed in U.S. Pat. No. 5,580,560; Factor VIIa that has been proteolytically cleaved between residues 290 and 291 or between residues 315 and 316; oxidized forms of Factor VIIa; L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII; L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V1158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII; S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V1158D-FVII, K316H/E296V-FVII, K316H/M298Q-FVII, K316H/V1158T-FVII, K316Q/L305V-FVII, K316Q/K337A-FVII, K316Q/V1 58D-FVII, K316Q/E296V-FVII, K316Q/M298Q-FVII, K316Q/V1 58T-FVII, S314E/L305V/K337A-FVII, S314E/L305V/V1158D-FVII, S314E/L305V/E296V-FVII, S314E/L305V/M298Q-FVII, S314E/L305V/V1158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V 158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A —FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/M296V-FVII, K316H/L305V/K337A/V158D-FVII, K316H/L305V/V158D/M298Q-FVII, K316H/L305V/V1158D/E296V-FVII, K316H/L305V/V1158T/M298Q-FVII, K316H/L305V/V1158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A —FVII, K316H/L305V/V158D/E296V/M298Q/K337A-FVII, K316H/L305V/V1158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V1158D/E296V-FVII, K316Q/L305V/V158T/M298Q-FVII, K316Q/L305V/V1158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V1158T/E296V/K337A-FVII, K316Q/L305V/V1158D/K337A/M298Q-FVII, K316Q/L305V/V158D/E296V/K337A —FVII, K316Q/L305V/V158D/E296V/M298Q/K337A-FVII, and K316Q/L305V/V158T/E296V/M298Q/K337A-FVII.

8. A method according to claim 1, wherein the subject suffers from haemophilia A.

9. A method according to claim 1, wherein the amount is between about 120-150 microg/kg bw of factor VIIIa or a corresponding amount of a factor VII-related polypeptide.

10. A method according to claim 1, wherein the naive subject is below 36 months of age.

11. A method according to claim 10, wherein the naive subject is below about 24 months of age,

12. A method for preventing formation of inhibitors to blood coagulation factor VIII, the method comprising administering to a naive subject in need of coagulation therapy (i) a first amount of a first hemostatic agent selected from the group consisting of factor VIIIa and a factor VII-related polypeptide and (ii) a second amount of a second hemostatic agent, wherein said first and second amounts together are effective for said coagulation therapy.

13. A method according to claim 12, wherein the second hemostatic agent is selected from the group consisting of: factor XII, factor V, PAI-1, factor XI, thrombomodulin, aprotinin, TAFI, a tPA-inhibitor, a TFPI-inhibitor, alpha2-antiplasmin, a protein C-inhibitor, a protein S-inhibitor, tranexamic acid, and epsilon-aminocaproic acid.

14. A method according to claim 13, wherein the first hemostatic agent is factor VIIIa and the second hemostatic agent is a factor VII-related polypeptide.

15. A method for preventing formation of inhibitors to blood coagulation factor IX, the method comprising administering to a naive subject in need of coagulation therapy a coagulation-effective amount of a polypeptide selected from the group consisting of factor VIIIa and a factor VII-related polypeptide.

16. A method according to claim 15, wherein said administering is via an intravenous, subcutaneous, intradermal, or intramuscular route.

17. A method according to claim 15, wherein the amount is at least about 120 microg/kg.

18. A method according to claim 17, wherein the amount is at least about 150 microg/kg factor VII or a corresponding amount of a factor VII-related polypeptide.

19. A method according to claim 15, wherein the polypeptide is human factor VIIIa.

20. A method according to claim 15, wherein the factor VIIIa-related polypeptide is a factor VIIa sequence variant.

21. A method according to claim 20, wherein the factor VIIa-related polypeptide is selected from the group consisting of: S52A-FVIIa, S60A-FVIIa, FVIIa variants exhibiting increased proteolytic stability as disclosed in U.S. Pat. No. 5,580,560; Factor VIIa that has been proteolytically cleaved between residues 290 and 291 or between residues 315 and 316; oxidized forms of Factor VIIa; L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII; L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V1158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII; S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, 5314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A —FVII, K316Q/L305V/V158D/E296V/M298Q/K337A-FVII, and K316Q/L305V/V158T/E296V/M298Q/K337A-FVII.

22. A method according to claim 15, wherein the subject suffers from haemophilia B.

23. A method according to claim 15, wherein the amount is between about 120-150 microg/kg bw of factor VIIIa or a corresponding amount of a factor VII-related polypeptide.

24. A method according to claim 15, wherein the naive subject is below 36 months of age.

25. A method according to claim 24, wherein the naive subject is below about 24 months of age,

26. A method for preventing formation of inhibitors to blood coagulation factor IX, the method comprising administering to a naive subject in need of coagulation therapy (i) a first amount of a first hemostatic agent selected from the group consisting of factor VIIIa and a factor VII-related polypeptide and (ii) a second amount of a second hemostatic agent, wherein said first and second amounts together are effective for said coagulation therapy.

27. A method according to claim 26, wherein the second hemostatic agent is selected from the group consisting of: factor XIII, factor V, PAI-1, factor XI, thrombomodulin, aprotinin, TAFI, a tPA-inhibitor, a TFPI-inhibitor, alpha2-antiplasmin, a protein C-inhibitor, a protein S-inhibitor, tranexamic acid, and epsilon-aminocaproic acid.

28. A method according to claim 27, wherein the first hemostatic agent is factor VIIIa and the second hemostatic agent is a factor VII-related polypeptide.
